(19) United States
(12) Patent Application Publication
(10) Pub. No.: US 2011/0060017 A1
(43) Pub. Date: Mar. 10, 2011

(54) NOVEL 1,2,3-TRIAZOLE DERIVATIVES USEFUL AS MODULATORS OF NICOTINIC ACETYLCHOLINE RECEPTORS

(75) Inventors: Antonio Nardi, Herzogenrath (DE); Jeppe Kejser Christensen, Kobenhavn (DK); Dan Peters, Malmo (SE)

(73) Assignee: Neurosearch A/S, Ballerup (DK)

(21) Appl. No.: 12/921,995

(22) PCT Filed: Mar. 9, 2009
(86) PCT No.: PCT/EP2009/052729

§ 371 Date: Nov. 19, 2010

Related U.S. Application Data
(60) Provisional application No. 61/035,927, filed on Mar. 12, 2008.

(30) Foreign Application Priority Data
Mar. 11, 2008 (DK) PA 2008 00370
Aug. 7, 2008 (DK) PA 2008 01078

(51) Int. Cl. A61K 31/4192; C07D 249/04; A61P 25/00; A61P 25/20; A61P 25/16; A61P 25/28; A61P 25/24; A61P 25/18; A61P 21/00; A61P 25/30; A61P 29/00; A61P 15/00; A61P 3/10; A61P 1/00

(52) U.S. Cl. 514/359; 548/255

(57) ABSTRACT

This invention relates to novel 1,2,3-triazole derivatives, which are found to be modulators of the nicotinic acetyl-choline receptors. Due to their pharmacological profile, the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### TECHNICAL FIELD

This invention relates to novel 1,2,3-triazole derivatives, which are found to be modulators of the nicotinic acetylcholine receptors. Due to their pharmacological profile, the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exert its biological effect via two types of cholinergic receptors, the muscarinic Acetyl Choline Receptors (mAChR) and the nicotinic Acetyl Choline Receptors (nAChR).

As it is well established that muscarinic acetylcholine receptors dominate quantitatively over nicotinic acetylcholine receptors in the brain area important to memory and cognition, and much research aimed at the development of agents for the treatment of memory related disorders have focused on the synthesis of muscarinic acetylcholine receptor modulators.

Recently, however, an interest in the development of nAChR modulators has emerged. Several diseases are associated with degeneration of the cholinergic system i.e. senile dementia of the Alzheimer type, vascular dementia and cognitive impairment due to the organic brain damage disease related directly to alcoholism.

### SUMMARY OF THE INVENTION

The present invention is devoted to the provision novel modulators of the nicotinic receptors, which modulators are useful for the treatment of diseases or disorders related to the cholinergic receptors, and in particular the nicotinic acetylcholine α7 receptor subtype.

The compounds of the invention may also be useful as diagnostic tools or monitoring agents in various diagnostic methods, and in particular for in vivo receptor imaging (neuroimaging), and they may be used in labelled or unlabelled form.

In its first aspect the invention provides 1,2,3-triazole derivatives of Formula I

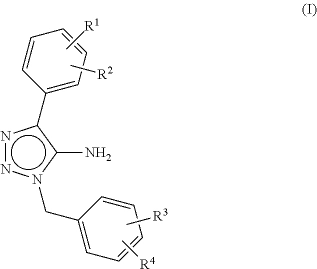

a stereoisomer thereof or a mixture of its stereoisomers, or a pharmaceutically acceptable salt thereof, wherein

R¹ and R² both represent halo, trifluoromethyl, trifluoromethoxy or cyano; and

R³ and R⁴, independently of each other, represent hydrogen, hydroxy, alkoxy or sulfamoyl; or

R¹ and R², independently of each other, represent hydrogen, hydroxy or alkoxy; and

R³ and R⁴ both represent halo, trifluoromethyl, trifluoromethoxy or cyano; or

one of R¹ and R² represents halo; and the other of R¹ and R² represents trifluoromethyl; and R³ and R⁴, independently of each other, represent hydrogen, hydroxy, alkoxy or sulfamoyl.

In a second aspect the invention provides pharmaceutical compositions comprising a therapeutically effective amount of the 1,2,3-triazole derivative of the invention, or a pharmaceutically acceptable addition salt thereof, together with at least one pharmaceutically acceptable carrier or diluent.

Viewed from another aspect the invention relates to the use of the 1,2,3-triazole derivative of the invention, or a pharmaceutically acceptable addition salt thereof, for the manufacture of pharmaceutical compositions/medicaments for the treatment, prevention or alleviation of a disease or a disorder or a condition of a mammal, including a human, which disease, disorder or condition is responsive to modulation of cholinergic receptors.

In yet another aspect the invention provides a method for treatment, prevention or alleviation of diseases, disorders or conditions of a living animal body, including a human, which disorder, disease or condition is responsive to modulation of cholinergic receptors, and which method comprises the step of administering to such a living animal body in need thereof a therapeutically effective amount of the 1,2,3-triazole derivative of the invention.

Other objects of the invention will be apparent to the person skilled in the art from the following detailed description and examples.

### DETAILED DISCLOSURE OF THE INVENTION

### 1,2,3-Triazole Derivatives

In its first aspect the invention provides 1,2,3-triazole derivatives of Formula I

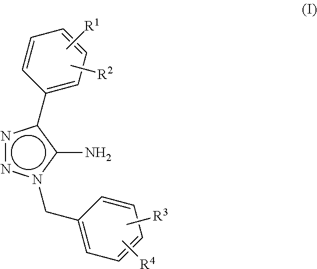

a stereoisomer thereof or a mixture of its stereoisomers, or a pharmaceutically acceptable salt thereof, wherein

R¹ and R² both represent halo, trifluoromethyl, trifluoromethoxy or cyano; and R³ and R⁴, independently of each other, represent hydrogen, hydroxy, alkoxy or sulfamoyl; or

R¹ and R², independently of each other, represent hydrogen, hydroxy, alkoxy; and R³ and R⁴ both represent halo, trifluoromethyl, trifluoromethoxy or cyano; or

one of R¹ and R² represents halo, and in particular fluoro; and the other of R¹ and R² represents trifluoromethyl; and R³ and R⁴, independently of each other, represent hydrogen, hydroxy, alkoxy or sulfamoyl.

In a preferred embodiment the 1,2,3-triazole derivative is a compound of Formula I, a stereoisomer thereof or a mixture of its stereoisomers, or a pharmaceutically acceptable salt thereof, wherein

R¹ and R² both represent halo, trifluoromethyl, trifluoromethoxy or cyano; and R³ and R⁴, independently of each other, represent hydrogen, hydroxy or alkoxy; or

R¹ and R², independently of each other, represent hydrogen, hydroxy or alkoxy; and R³ and R⁴ both represent halo, trifluoromethyl, trifluoromethoxy or cyano.

In another preferred embodiment the 1,2,3-triazole derivative of the invention is a compound of Formula I, a stereoisomer thereof or a mixture of its stereoisomers, or a pharmaceutically acceptable salt thereof, wherein R¹ and R² both represent halo, trifluoromethyl, trifluoromethoxy or cyano; and R³ and R⁴, independently of each other, represent hydrogen, hydroxy, alkoxy or sulfamoyl.

In a more preferred embodiment R¹ and R² both represent halo, trifluoromethyl, trifluoromethoxy or cyano; and R³ and R⁴, independently of each other, represent hydrogen, hydroxy or alkoxy.

In another more preferred embodiment R¹ and R² both represent halo, trifluoromethyl, trifluoromethoxy or cyano; R³ represent hydrogen; and R⁴ hydroxy or alkoxy, and in particular methoxy.

In a third more preferred embodiment R¹ and R² both represent halo or trifluoromethyl; R³ represent hydrogen; and R⁴ hydroxy or alkoxy, and in particular methoxy.

In a fourth more preferred embodiment R¹ and R² both represent halo, and in particular chloro; and R³ represent hydrogen; and R⁴ hydroxy or alkoxy, and in particular methoxy.

In a third preferred embodiment the 1,2,3-triazole derivative of the invention is a compound of Formula I, a stereoisomer thereof or a mixture of its stereoisomers, or a pharmaceutically acceptable salt thereof, wherein R¹ and R², independently of each other, represent hydrogen, hydroxy or alkoxy; and R³ and R⁴ both represent halo, trifluoromethyl, trifluoromethoxy or cyano.

In a more preferred embodiment R¹ represents hydrogen; R² represents hydroxy or alkoxy, and in particular methoxy; and R³ and R⁴ both represent halo, trifluoromethyl, trifluoromethoxy or cyano.

In another more preferred embodiment R¹ represents hydrogen; R² represents hydroxy or alkoxy, and in particular methoxy; and R³ and R⁴ both represent halo or trifluoromethyl.

In a third more preferred embodiment R¹ represents hydrogen; R² represents hydroxy or alkoxy, and in particular methoxy; and R³ and R⁴ both represent halo, and in particular chloro.

In a fourth preferred embodiment the 1,2,3-triazole derivative of the invention is a compound of Formula I, a stereoisomer thereof or a mixture of its stereoisomers, or a pharmaceutically acceptable salt thereof, wherein one of R¹ and R² represents halo, and in particular fluoro; and the other of R¹ and R² represents trifluoromethyl; and R³ and R⁴, independently of each other, represent hydrogen, hydroxy, alkoxy or sulfamoyl.

In a more preferred embodiment one of R¹ and R² represents halo, and in particular fluoro; and the other of R¹ and R² represents trifluoromethyl; and R³ and R⁴, independently of each other, represent hydrogen, hydroxy or sulfamoyl.

In a most preferred embodiment the 1,2,3-triazole derivative of the invention is
5-(2,4-Dichloro-phenyl)-3-(4-methoxy-benzyl)-3H-[1,2,3]triazol-4-ylamine;
3-(2,4-Dichloro-benzyl)-5-(4-methoxy-phenyl)-3H-[1,2,3]triazol-4-ylamine;
4-[5-Amino-1-(2,4-dichloro-benzyl)-1H-[1,2,3]triazol-4-yl]-phenol;
4-[5-Amino-4-(2,4-dichloro-phenyl)-[1,2,3]triazol-1-ylmethyl]-phenol;
4-[5-Amino-4-(2-fluoro-4-trifluoromethyl-phenyl)-[1,2,3]triazol-1-ylmethyl]-benzenesulfonamide;
4-[5-Amino-4-(2,4-dichloro-phenyl)-[1,2,3]triazol-1-ylmethyl]-benzenesulfonamide;
5-(2-Fluoro-4-trifluoromethyl-phenyl)-3-(4-methoxy-benzyl)-3H-[1,2,3]triazol-4-ylamine; or
4-[5-Amino-4-(2-fluoro-4-trifluoromethyl-phenyl)-[1,2,3]triazol-1-ylmethyl]-phenol;

a stereoisomer thereof or a mixture of its stereoisomers, or a pharmaceutically acceptable salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O—” group, wherein alkyl is as defined above. Examples of preferred alkoxy groups of the invention include methoxy and ethoxy.

### Pharmaceutically Acceptable Salts

The 1,2,3-triazole derivative of the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a 1,2,3-triazole derivative of the invention include alkali metal salts, such as the sodium salt of a compound of the invention containing a carboxy group.

### Steric Isomers

It will be appreciated by those skilled in the art that the 1,2,3-triazole derivatives of the present invention may exist in different stereoisomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Methods of Producing 1,2,3-Triazole Derivatives

The 1,2,3-triazole derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The present invention is devoted to the provision novel modulators of the nicotinic receptors, which modulators are useful for the treatment of diseases or disorders related to the cholinergic receptors, and in particular the nicotinic acetylcholine receptor (nAChR). Preferred compounds of the invention show activity as positive modulators of the nicotinic acetylcholine α7 receptor subtype.

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

The compounds of the invention may also be useful as diagnostic tools or monitoring agents in various diagnostic methods, and in particular for in vivo receptor imaging (neuroimaging), and they may be used in labelled or unlabelled form.

In a preferred embodiment the disease, disorder or condition contemplated according to the invention, and responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudodementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated withdrawal symptoms caused by termination of use of addictive substances, including nicotine withdrawal symptoms, opioid withdrawal symptoms including heroin, cocaine and morphine, benzodiazepine withdrawal symptoms including benzodiazepine-like drugs and alcohol.

In a more preferred embodiment the disease, disorder or condition responsive to modulation of nicotinic acetylcholine receptors is a cognitive disorder, psychosis, schizophrenia or depression.

In another more preferred embodiment the disease, disorder or condition responsive to modulation of nicotinic acetylcholine receptors is associated with smooth muscle contractions, including convulsive disorders, angina pectoris, premature labour, convulsions, diarrhoea, asthma, epilepsy, tardive dyskinesia, hyperkinesia, premature ejaculation and erectile difficulty.

In still another more preferred embodiment the disease, disorder or condition responsive to modulation of nicotinic acetylcholine receptors is related to the endocrine system, such as thyrotoxicosis and pheochromocytoma.

In yet another more preferred embodiment the disease, disorder or condition responsive to modulation of nicotinic acetylcholine receptors is a neurodegenerative disorder including transient anoxia and induced neuro-degeneration.

In a further more preferred embodiment the disease, disorder or condition responsive to modulation of nicotinic acetylcholine receptors is pain, including mild, moderate or even severe pain of acute, chronic or recurrent character, as well as pain caused by migraine, postoperative pain, and phantom limb pain. The pain may in particular be neuropathic pain, chronic headache, central pain, pain related to diabetic neuropathy, to postherpetic neuralgia, or to peripheral nerve injury.

In a further more preferred embodiment the disease, disorder or condition responsive to modulation of nicotinic acetylcholine receptors is an inflammatory skin disorder such as acne and rosacea, Crohn's disease, inflammatory bowel disease, ulcerative colitis, and diarrhoea.

Finally the compounds of the invention may be useful for the treatment of withdrawal symptoms caused by termination of use of addictive substances. Such addictive substances include nicotine containing products such as tobacco, opioids such as heroin, cocaine and morphine, benzodiazepines and benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In this context “treatment” covers treatment, prevention, prophylactics and alleviation of withdrawal symptoms and abstinence as well as treatment resulting in a voluntary diminished intake of the addictive substance.

### Pharmaceutical Compositions

In another aspect the invention provides novel pharmaceutical compositions comprising a therapeutically effective amount of 1,2,3-triazole derivative of the invention.

While a 1,2,3-triazole derivative of the invention for use in therapy may be administered in the form of the raw compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the 1,2,3-triazole derivative of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

The 1,2,3-triazole derivatives of the present invention are valuable nicotinic receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors, and which method comprises administering to such a living animal body, including a human, in need thereof an effective amount of a 1,2,3-triazole derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### BRIEF DESCRIPTION OF THE DRAWING

The present invention is further illustrated by reference to the accompanying drawing, in which FIGS. 1A and 1B show the modulatory effect of Compound 4 (i.e. 4-[5-Amino-4-(2,4-dichloro-phenyl)-[1,2,3]triazol-1-ylmethyl]-phenol) on acetylcholine currents induced in nAChR α7 receptors expressed in Xenopus oocytes:

FIG. 1A shows current traces induced by 100 μM acetylcholine in the absence and in the presence of 0.01 to 31.6 μM of Compound 4;

FIG. 1B shows the concentration-response relationship for the positive modulation of 100 μM acetylcholine responses induced by Compound 4; i.e. % modulation of control vs. log [c] (M). The calculated EC₅₀-value is 1.1 μM and the maximal modulation of the acetylcholine response is 277%.

### EXAMPLES

The invention is further illustrated with reference to the following examples, which are not intended to be in any way limiting to the scope of the invention as claimed.

### Example 1

### Preparatory Example

### General Experimental Procedure

Chemical synthesis of the 1,2,3-triazole derivatives of the invention is envisioned by the use of suitably-substituted benzylazides. These were prepared from the correspondent commercial benzylchlorides or benzylbromides by reaction with sodium azide. As an example of this synthetic method, we report the detailed experimental procedure for the synthesis of intermediate 1 (INT-1). When the suitable benzylchlorides or benzylbromides were not commercially available, they were prepared from the correspondent benzylalcohols, upon the well-know treatment with oxalyl chloride. Other benzylchlorides or bromides, which are not commercially-available, can be obtained by known methods described in the literature. As an example of these latter methods, the synthesis of INT-2 is reported. The resulting benzylazides finally underwent a base-catalysed and regiospecific condensation with proper activated methylenic compounds, i.e. (substituted or not) commercial phenylacetonitrile derivatives. In presence of methyl ether substituent(s), ether cleavage followed after the cycloaddition, upon mild nucleophilic substitution with the Lewis acid boron tribromide.

### 1-Azidomethyl-4-methoxy-benzene (Intermediate compound, INT-1)

To a solution of commercial 4-methoxybenzyl chloride (2.50 g, 15.963 mmol, 1 eq) in ethanol (50 ml) and water (4 ml) mixture, an excess of sodium azide (10.3 g, 159.63 mmol, 10 eq) is added and the suspension was refluxed for 24 h. The resulting reaction mixture is filtered (to remove inorganic salts) and the solid recovered is washed with ethanol. The filtrate is concentrated and the ethanol solution residue diluted with water and extracted with dichloromethane. The combined organic layers are washed with brine, dried over MgSO₄, filtered and evaporated to dryness, to afford 2.7 g of colourless liquid, which is used as such for the next step. Infra red spectra (cm⁻¹) (N3): 2096.

### 4-Azidomethyl-benzenesulfonamide (INT-2)

To a stirred solution of 4-bromomethyl-benzenesulfonamide (4.500 g, 17.9917 mmol) (prepared from 4-bromomethyl-benzenesulfonyl chloride upon treatment with ammonia (as described by Yee Y K et al. in Journal of Medicinal Chemistry 1990 33 (9) 2437-2451) in N,N-dimethylformamide (30 ml), sodium azide is added (11.696 g, 179.917 mmol) and the reaction mixture is heated at 90° C. for 14 hours under a nitrogen atmosphere. The suspension is filtered, to remove the excess of sodium azide, and the solid residue is washed with ethyl acetate (4×50 ml). The mother liquids are concentrated, to afford a crude yellow liquid (3.820 g, 100% mass balance), that is used as such for the next step. IR: 2098.8 cm⁻¹.

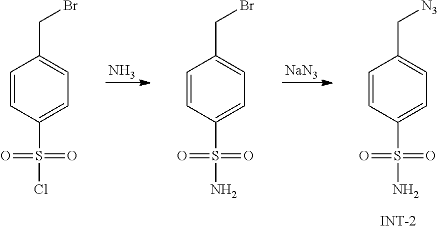

### 5-(2,4-Dichloro-phenyl)-3-(4-methoxy-benzyl)-3H-[1,2,3]triazol-4-ylamine (Compound 1)

To a stirred and ice-cooled solution of 1-azidomethyl-4-methoxy-benzene (0.500 g, 3.064 mmol, 1 eq) and commercial 2,4-dichlorobenzyl cyanide (0.684 g, 3.677 mmol, 1.2 eq) in ethanol (30 ml), an ice-cooled solution of sodium methoxide (0.248 g, 4.596 mmol, 1.5 eq) in ethanol (15 ml) is added drop-wise. The reaction mixture is allowed to attain room temperature and stirred overnight. The resulting mixture is evaporated to dryness and the crude residue is dissolved in dichloromethane and washed with brine, dried over MgSO₄, filtered and evaporated to dryness again, to afford 1.1 g of crude compound as brownish gum. This crude material is purified by flash chromatography eluting with 26% ethyl acetate in hexane, to get 0.225 g of the title compound as a white solid (yield: ˜24%). LC-ESI-HRMS of [M+H]+ shows 349.061 Da. Calc. 349.062292 Da, dev. −3.7 ppm. M.p. 134.4-136.8° C.

### 3-(2,4-Dichloro-benzyl)-5-(4-methoxy-phenyl)-3H-[1,2,3]-triazol-4-ylamine (Compound 2)

To a stirred and ice-cooled solution of 1-azidomethyl-2,4-dichloro-benzene (0.500 g, 2.4747 mmol, 1 eq) and 4-methoxyphenylacetonitrile (0.4506 g, 2.9696 mmol, 1.2 eq) in ethanol (15 ml), a freshly prepared and ice-cooled solution of sodium methoxide (0.200 g, 3.712 mmol, 1.5 eq) in methanol (15 ml) is added drop-wise over a period of 20 min. The resulting reaction mixture is allowed to attain room temperature, stirred overnight and evaporated to dryness, to afford 0.940 g of a brown semi-solid residue. This crude residue is purified by column chromatography eluting with 5-30% ethyl acetate in hexane, to afford 0.175 g of the title compound as a brownish solid (yield: 20%). LC-ESI-HRMS of [M+H]+ shows 349.0605 Da. Calc. 349.062292 Da, dev. −5.1 ppm. M.p. 156.5-159° C.

### 4-[5-Amino-1-(2,4-dichloro-benzyl)-1H-[1,2,3]triazol-4-yl]-phenol (Compound 3)

To a stirred and ice-cooled solution of 3-(2,4-dichloro-benzyl)-5-(4-methoxy-phenyl)-3H-[1,2,3]triazol-4-ylamine (0.200 g, 0.5727 mmol, 1 eq) in anhydrous dichloromethane (10 ml), cooled to −78° C. and kept under nitrogen, a solution of boron tribromide (1.00 g, 4.0089 mmol, 7 eq) in anhydrous dichloromethane (10 ml) is added drop-wise. The reaction mixture is allowed to attain room temperature spontaneously and stirred overnight. The mixture is then cooled again in an ice-salt bath and the excess of the reagent is decomposed by treatment with methanol (5 ml) followed by water (10 ml). The organic layer is washed with water and extracted with 10% NaOH. Acidification of the alkaline extract provides a precipitate that is collected by filtration (0.220 g). This latter product is purified by column chromatography eluting with 30% ethyl acetate in hexane, to afford the title compound (0.120 g) as a light brown solid (yield 62%). LC-ESI-HRMS of [M+H]+ shows 335.0481 Da. Calc. 335.046642 Da, dev. 4.4 ppm.

### 4-[5-Amino-4-(2,4-dichloro-phenyl)-[1,2,3]triazol-1-ylmethyl]-phenol (Compound 4)

To a solution of 5-(2,4-dichloro-phenyl)-3-(4-methoxy-benzyl)-3H-[1,2,3]triazol-4-ylamine (0.500 g, 1.4318 mmol, 1 eq) in anhydrous dichloromethane (20 ml), cooled to −78° C. and kept under nitrogen, a solution of boron tribromide (2.51 g, 10.0226 mmol, 7 eq) in anhydrous dichloromethane (10 ml) is added drop-wise. The reaction mixture is allowed to attain room temperature spontaneously and stirred overnight. The mixture is then cooled again in an ice-salt bath and the excess of the reagent is decomposed by treatment with methanol (10 ml) followed by water (15 ml). The organic layer is washed with water and extracted with 10% NaOH. Acidification of the alkaline extract provides a precipitate that was collected by filtration (0.430 g). This latter product is purified by column chromatography eluting with 40% ethyl acetate in hexane, to afford the title compound (0.300 g) as a white solid (yield 62%). LC-ESI-HRMS of [M+H]+ shows 335.0478 Da. Calc. 335.046642 Da, dev. 3.5 ppm M.p. 172.5-174.1° C.

### 4-[5-Amino-4-(2-fluoro-4-trifluoromethyl-phenyl)-[1,2,3]-triazol-1-ylmethyl]-benzenesulfonamide (Compound 5)

To a stirred and ice-cooled solution of commercial 2-fluoro-4-(trifluoromethyl)phenyl acetonitrile (2.297 g, 11.3083 mmol) and 4-azidomethyl-benzenesulfonamide (INT-2) (2.000 g, 9.4236 mmol) in methanol (20 ml), sodium methoxide (0.7636 g, 14.1354 mmol) is added portion wise and the reaction mixture is allowed to attain room temperature spontaneously and stirred at this temperature for additional 70 hours. The resulting reaction mixture is evaporated to dryness and the crude residue is purified by preparative elution chromatography, to afford 0.055 g (1.5% yield) of the title compound. M.p. 180.1-181.3° C.

### 4-[5-Amino-4-(2,4-dichloro-phenyl)-[1,2,3]-triazol-1-ylmethyl]-benzenesulfonamide (Compound 6)

To a stirred and ice-cooled solution of commercial 2,4-dichlorobenzyl cyanide (1.893 g, 10.1776 mmol) and 4-azidomethyl-benzenesulfonamide (INT-2) (1.800 g, 8.4813 mmol) in methanol (20 ml), sodium methoxide (0.7636 g, 14.1354 mmol) is added portion wise and the reaction mixture is allowed to attain room temperature spontaneously and stirred at this temperature for additional 54 hrs. The resulting reaction mixture is evaporated to dryness and the crude residue is purified by preparative elution chromatography, to afford 0.447 g (12% yield) of the title compound. M.p. 204-205.6° C.

### 5-(2-Fluoro-4-trifluoromethyl-phenyl)-3-(4-methoxy-benzyl)-3H-[1,2,3]-triazol-4-ylamine (Compound 7)

To a stirred and ice-cooled solution of 2-fluoro-4-(trifluoromethyl)phenyl acetonitrile (1.494 g, 7.3538 mmol) and 1-azidomethyl-4-methoxy-benzene (1.000 g, 6.1282 mmol) (prepared as described by Compain-Batissou M in Heterocycles 2007 71 (1) 27-38) in methanol (40 ml), sodium methoxide (0.497 g, 9.1922 mmol) is added portion wise and the mixture is allowed to attain room temperature spontaneously and stirred for additional 60 hours. The resulting reaction mixture is concentrated, added water, and extracted with dichloromethane (3×80 ml). The combined organic layers are washed with brine (15 ml), dried over MgSO₄, filtered and concentrated to give a crude brown liquid residue (2.240 g, 100% mass balance). This crude material is purified by column chromatography over silica gel (60-120 mesh) eluting with 25% ethyl acetate in hexane, to afford the title compound as an off-white solid (1.010 g, 45% yield). M.p. 153.2-154.4° C. LC-ESI-HRMS of [M+H]+ shows 367.1174 Da. Calc. 367.118198 Da, dev. −2.2 ppm.

### 4-[5-Amino-4-(2-fluoro-4-trifluoromethyl-phenyl)-[1,2,3]-triazol-1-ylmethyl]-phenol (Compound 8)

To a stirred solution of 5-(2-fluoro-4-trifluoromethyl-phenyl)-3-(4-methoxy-benzyl)-3H-[1,2,3]triazol-4-ylamine (Compound 7) (0.450 g, 1.2284 mmol) in anhydrous dichloromethane (35 ml), cooled to −78° C. and under a nitrogen flow, a solution of boron tribromide (2.154 g, ˜0.81 ml, 8.5988 mmol) in 5 ml of anhydrous dichloromethane is added drop-wise. The mixture is allowed to reach room temperature spontaneously overnight, cooled again in an ice-salt bath, and the excess of the reagent is decomposed upon drop-wise addition of 10 ml of methanol and 15 ml of water. After 5 min stirring, 10% sodium hydroxide solution (15 ml) is added and the aqueous layer, once separated, is acidified with 10% hydrochloric acid solution and extracted with chloroform (3×80 ml). The combined organic layers are dried over MgSO₄, filtered and evaporated to afford a solid residue (0.410 g). This is purified by column chromatography over silica gel (60-120 mesh) eluting with 35% ethyl acetate in hexane, to obtain the title compound as an off-white solid (0.255 g, 57% yield). M.p. 168.2-169.5° C. LC-ESI-HRMS of [M+H]+ shows 353.1021 Da. Calc. 353.102548 Da, dev. −1.3 ppm.

### Example 2

### Biological Activity

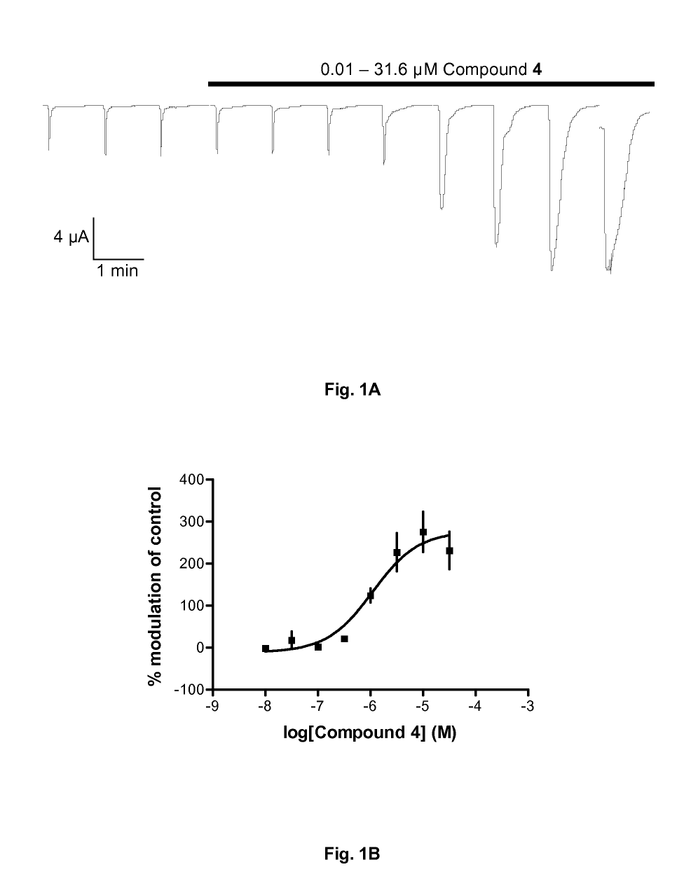

In this example the positive modulation of wild-type nAChR α7 receptors by Compound 4 (i.e. 4-[5-Amino-4-(2,4-dichloro-phenyl)-[1,2,3]triazol-1-ylmethyl]phenol; FIGS. 1A and 1B) was determined using nAChR α7 receptors heterologously expressed in Xenopus laevis oocytes.

The electrical current through the nAChR α7 channel was measured using conventional two-electrode voltage clamp and nAChR α7 currents were activated by applying pulses of agonist-containing solution onto the nAChR α7 expressing oocyte.

In brief, the oocytes were placed in a recording chambers and continuously super-fused with an Oocyte Ringer (OR) solution containing 90 mM NaCl, 2.5 mM KCl, 2.5 mM CaCl₂, 1 mM MgCl₂ and 5 mM HEPES (pH adjusted to 7.4). The oocytes were clamped at −60 mV and currents were induced by applying 20 s pulses of 100 μM acetylcholine dissolved in OR. The intervals between the acetylcholine applications were 5 minutes, during which the oocytes were washed with OR. The first three applications were control applications to insure a constant response level of 100 μM acetylcholine. For the subsequent 8 test applications, increasing concentrations (0.01-31.6 μM) of Compound 4 was applied 30 s before and during the acetylcholine (100 μM) application, which caused a robust increase in the acetylcholine-induced current amplitude.

The positive modulation in the presence of Compound 4 was calculated as (test-control)/control*100% and the concentration response curve for this positive modulation was fitted to the sigmoidal logistic equation: I=Iₘₐₓ/(1+(EC₅₀/[compound])ⁿ), where Iₘₐₓ represents the maximal modulation of the control response, EC₅₀ is the concentration causing a half maximal response, and n is the slope coefficient.

The calculated EC₅₀ value and Iₘₐₓ value for Compound 4 were 1.1 μM and 277%, respectively.

1. A 1,2,3-triazole derivative represented by Formula I 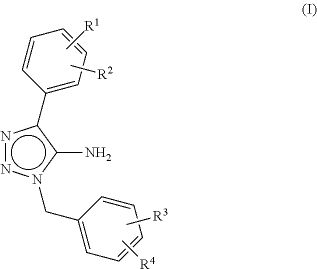
a stereoisomer thereof or a mixture of its stereoisomers, or a pharmaceutically acceptable salt thereof, wherein
R¹ and R² both represent halo, trifluoromethyl, trifluoromethoxy or cyano; and
R³ and R⁴, independently of each other, represent hydrogen, hydroxy, alkoxy or sulfamoyl; or
R¹ and R², independently of each other, represent hydrogen, hydroxy or alkoxy; and
R³ and R⁴ both represent halo, trifluoromethyl, trifluoromethoxy or cyano; or
one of R¹ and R² represents halo; and the other of R¹ and R² represents trifluoromethyl; and R³ and R⁴, independently of each other, represent hydrogen, hydroxy, alkoxy or sulfamoyl.

2. The 1,2,3-triazole derivative of claim 1, a stereoisomer thereof or a mixture of its stereoisomers, or a pharmaceutically acceptable salt thereof, wherein
R¹ and R² both represent halo, trifluoromethyl, trifluoromethoxy or cyano; and
R³ and R⁴, independently of each other, represent hydrogen, hydroxy, alkoxy or sulfamoyl.

3. The 1,2,3-triazole derivative of claim 1, a stereoisomer thereof or a mixture of its stereoisomers, or a pharmaceutically acceptable salt thereof, wherein
R¹ and R², independently of each other, represent hydrogen, hydroxy or alkoxy; and
R³ and R⁴ both represent halo, trifluoromethyl, trifluoromethoxy or cyano.

4. The 1,2,3-triazole derivative of claim 1, a stereoisomer thereof or a mixture of its stereoisomers, or a pharmaceutically acceptable salt thereof, wherein
one of R¹ and R² represents halo; and
the other of R¹ and R² represents trifluoromethyl; and
R³ and R⁴, independently of each other, represent hydrogen, hydroxy, alkoxy or sulfamoyl.

5. The 1,2,3-triazole derivative of claim 1, which is
5-(2,4-Dichloro-phenyl)-3-(4-methoxy-benzyl)-3H-[1,2,3]triazol-4-ylamine;
3-(2,4-Dichloro-benzyl)-5-(4-methoxy-phenyl)-3H-[1,2,3]triazol-4-ylamine;
4-[5-Amino-1-(2,4-dichloro-benzyl)-1H-[1,2,3]triazol-4-yl]-phenol;
4-[5-Amino-4-(2,4-dichloro-phenyl)-[1,2,3]triazol-1-ylmethyl]-phenol;
4-[5-Amino-4-(2-fluoro-4-trifluoromethyl-phenyl)-[1,2,3]triazol-1-ylmethyl]-benzenesulfonamide;
4-[5-Amino-4-(2,4-dichloro-phenyl)-[1,2,3]triazol-1-ylmethyl]-benzenesulfonamide;
5-(2-Fluoro-4-trifluoromethyl-phenyl)-3-(4-methoxy-benzyl)-3H-[1,2,3]triazol-4-ylamine; or
4-[5-Amino-4-(2-fluoro-4-trifluoromethyl-phenyl)-[1,2,3]triazol-1-ylmethyl]-phenol;
a stereoisomer thereof or a mixture of its stereoisomers, or a pharmaceutically acceptable salt thereof.

6. A pharmaceutical composition comprising a therapeutically effective amount of a 1,2,3-triazole derivative of any one of claims 1-5, a stereoisomer thereof or a mixture of its stereoisomers, or a pharmaceutically acceptable addition salt thereof, or a prodrug thereof, together with at least one pharmaceutically acceptable carrier or diluent.

7. A 1,2,3-triazole derivative of any one of claims 1-5, a stereoisomer thereof or a mixture of its stereoisomers, or a pharmaceutically acceptable addition salt thereof, for use as a medicament.

8-10. (canceled)

11. A method of treatment, prevention or alleviation of a disease or a disorder or a condition of a living animal body, including a human, which disorder, disease or condition is responsive to modulation of nicotinic acetylcholine receptors, which method comprises the step of administering to such a living animal body in need thereof a therapeutically effective amount of a 1,2,3-triazole derivative of any one of claims 1-5, a stereoisomer thereof or a mixture of its stereoisomers, or a pharmaceutically acceptable salt thereof.

12. The method according to claim 11, wherein the disease, disorder or condition responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudodementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated withdrawal symptoms caused by termination of use of addictive substances, including nicotine withdrawal symptoms, opioid withdrawal symptoms, including heroin, cocaine and morphine, benzodiazepine withdrawal symptoms including benzodiazepine-like drugs and alcohol.
